(19) United States
(12) Patent Application Publication
(10) Pub. No.: US 2009/0177087 A1
(43) Pub. Date: Jul. 9, 2009

(54) ULTRASONIC IMAGING APPARATUS

(76) Inventors: Sei Kato, Tokyo (JP); Tadashi Shimazaki, Tokyo (JP); Hiroshi Hashimoto, Tokyo (JP)

Correspondence Address: Patrick W. Rasche;Armstrong Teasdale LLP, One Metropolitan Square, Suite 2600, St. Louis, MO, 63102, US

(21) Appl. No.: 12/147,424

(22) Filed: Jun. 26, 2008

(30) Foreign Application Priority Data
Jun. 28, 2007 (JP) 2007-169928

(51) Int. Cl. A61B 8/14

(52) U.S. Cl. 600/443

(57) ABSTRACT

An ultrasonic imaging apparatus performs a plurality of times an action to transmit ultrasonic pulses to a subject, receives said plurality of times the reflected ultrasonic pulse train of said ultrasonic pulses reflected from said subject, generates one line of sound ray information by using said plurality of reflected ultrasonic pulse trains, and forms image information in which items of said sound ray information differing in the position or the direction of said transmission and said reception are arrayed. Said ultrasonic imaging apparatus includes: a B mode processing unit which forms B mode image information by using any one of said plurality of reflected ultrasonic pulse trains; a kinetic information detecting unit which detects kinetic information on said subject on the basis of said B mode image information; and a motion compensating unit which compensates image information for motions on the basis of said kinetic information.

### CROSS REFERENCE TO RELATED APPLICATIONS

This application claims the benefit of Japanese Patent Application No. 2007-169928 filed Jun. 28, 2007, which is hereby incorporated by reference in its entirety.

### BACKGROUND OF THE INVENTION

The subject matter disclosed herein relates to an ultrasonic imaging apparatus which transmits ultrasonic pulses to a subject a plurality of times, receives a reflected ultrasonic pulse train a plurality of times, and generates one line of sound ray information by using this plurality of reflected ultrasonic pulse trains.

Over the recent years, it has become a common practice to administer a contrast medium to a subject and to observe with an ultrasonic imaging apparatus a blood stream or a tissue that contains this contrast medium and is depicted in high luminance. For this observation, a contrast imaging mode is selected for clear recognition of the contrast medium. In a contrast imaging mode, for instance an imaging method known as a contrast harmonic B mode using a pulse inversion system is applied. By the contrast harmonic B mode, ultrasonic pulses similar in shape but differing in phase by 180 degrees are alternately transmitted to the subject, two reflected ultrasonic pulse trains differing in phase by 180 degrees from the subject are received and, after adding the pulse trains, a contrast harmonic B mode image is displayed.

In the contrast harmonic B mode image, which derives from extraction of only the nonlinear response part of the reflected ultrasonic pulse trains, the pixel value of the tissue part image of the subject is substantially zero, but on the other hand the contrast medium part image is depicted in high luminance (see Japanese Unexamined Patent Publication No. 2004-147823, for example).

The ultrasonic imaging apparatus also acquires contrast harmonic B mode images in an imaging area in which a contrast medium is present successively in a time series, and synthesizes these images. By this synthetic method known as Cine Capture/Accumulation processing, pixel values in the same pixel positions of this image information varying over time are compared, and a synthetic image is formed by using the larges pixel for each position among the compared pixel values as the new pixel value

However, according to the above-cited case of the background art, synthesized images formed by Cine Capture/Accumulation processing may turn out to be blurred images. Thus, contrast harmonic B mode images acquired in the contrast harmonic B mode include bodily motions including pulsation, and the tissue part may more or less vary in position from one image to next. Synthesized images formed by synthesizing pieces of image information involving such positional deviations may turn out to be blurred.

Attempts are also made to correct positional deviations in contrast harmonic B mode images and thereby to prevent synthesized images from blurring. However, contrast harmonic B mode images are images in which tissue signals are suppressed, and it is difficult to detect bodily motions from them. Therefore, the correction of positional deviations in image information cannot be accurate, and the blurring of synthesized images cannot be reduced.

Another practice is to acquire B mode images in which the tissue part is more clearly imaged separately from contrast harmonic B mode images, accurately detect bodily motions from these B mode images and correct the positional deviations in the contrast harmonic B mode images. This method, however, reduces the time available for acquiring the contrast harmonic B mode images by the time spent on the acquisition of the B mode images, and constitutes a factor to bring down the frame rate of the contrast harmonic B mode images. This drop in the real time availability of this contrast harmonic B mode image is particularly undesirable when variations over time in the contrast medium that flows together with the blood stream are to be observed.

Therefore, it is desirable that an ultrasonic imaging apparatus that can reliably prevent contrast harmonic B mode images from deviating in position without affecting the real time availability of contrast harmonic B mode images and accordingly to prevent synthesized images using the contrast harmonic B mode images from becoming blurred is realized.

### BRIEF DESCRIPTION OF THE INVENTION

It is desirable that the problems described previously are solved.

An ultrasonic imaging apparatus according to a first aspect of the invention performs a plurality of times an action to transmit ultrasonic pulses to a subject, receives the plurality of times the reflected ultrasonic pulse train of the ultrasonic pulses reflected from the subject, generates one line of sound ray information by using the plurality of reflected ultrasonic pulse trains, and forms image information in which items of the sound ray information differing in the position or the direction of the transmission and the reception are arrayed, the ultrasonic imaging apparatus being provided with: a B mode processing unit which forms B mode image information by using any one of the plurality of reflected ultrasonic pulse trains; a kinetic information detecting unit which detects kinetic information on the subject on the basis of the B mode image information; and a motion compensating unit which compensates image information for motions on the basis of the kinetic information.

According to this first aspect of the invention, the ultrasonic imaging apparatus with its B mode processing unit forms B mode image information by using any one of the plurality of reflected ultrasonic pulse trains, with its kinetic information detecting unit detects kinetic information on the subject on the basis of the B mode image information, and with its motion compensating unit compensates image information for motions on the basis of the kinetic information.

An ultrasonic imaging apparatus according to a second aspect of the invention, after performing a first transmitting/receiving action to transmit ultrasonic pulses to a subject and receive the reflected ultrasonic pulse train of the first ultrasonic pulses reflected from the subject and a second transmitting/receiving action to transmit inversion pulses similar in shape but differing in phase by 180 degrees from the ultrasonic pulses and receive the reflected inversion pulse train of the inversion pulse reflected from the subject, generates one line of sound ray information on the basis of the reflected ultrasonic pulse train and the reflected inversion pulse train, and forms image information in which items of the sound ray information differing in the position or the direction of the first and second transmitting/receiving actions are arrayed, the ultrasonic imaging apparatus being provided with: a B mode processing unit which forms B mode image information by using the reflected ultrasonic pulse train or the reflected inversion pulse train; a kinetic information detecting unit which detects kinetic information on the subject on the basis of the B mode image information; and a motion compensating unit which compensates image information for motions on the basis of the kinetic information.

According to this second aspect of the invention, the ultrasonic imaging apparatus with its B mode processing unit forms B mode image information by using the reflected ultrasonic pulse train or the reflected inversion pulse train, with its kinetic information detecting unit detects kinetic information on the subject on the basis of the B mode image information, and with its motion compensating unit compensates image information for motions on the basis of the kinetic information.

In the ultrasonic imaging apparatus according to a third aspect of the invention, in the second aspect, the first transmitting/receiving action and the second transmitting/receiving action in the second aspect are performed a plurality of times each to generate the one line of sound ray information.

According to this third aspect of the invention, image information of a high S/N ratio is acquired.

In the ultrasonic imaging apparatus according to a fourth aspect of the invention, in the second or third aspect the sound ray information is generated by adding the receiving signal of the first transmitting/receiving action and the second transmitting/receiving action.

According to this fourth aspect of the invention, only linear response parts are extracted from the reflected ultrasonic pulses and the reflected inversion pulses.

The ultrasonic imaging apparatus according to a fifth aspect of the invention, the ultrasonic imaging apparatus in any of the first through fourth aspects is further provided with synthesized image forming unit which compares pixel values in the same pixel position in a plurality of sets of image information formed in a time sequence, and forms synthesized image information consisting of the highest of the pixel value.

According to this fifth aspect of the invention, how the image of image information varies over time is traced in a single synthesized image of synthesized image information.

In the ultrasonic imaging apparatus according to a sixth aspect of the invention, in the fifth aspect the synthesized image forming unit forms the synthesized image information by using image information having undergone the motion compensation.

According to this sixth aspect of the invention, the synthesized image is prevented from being subjected to blurring.

In the ultrasonic imaging apparatus according to a seventh aspect of the invention, the ultrasonic imaging apparatus in any of the first through sixth aspects is further provided with a memory unit which stores, in a time series, the image information together with time information that has been formed.

According to this seventh aspect of the invention, motion compensation is performed by using past image information.

In the ultrasonic imaging apparatus according to an eighth aspect of the invention, in any of the first through seventh aspects, the kinetic information detecting unit detects the kinetic information on the tissue part image of the subject contained in the B mode image of the B mode image information.

According to this eighth aspect of the invention, reliable kinetic information is detected.

In the ultrasonic imaging apparatus according to a ninth aspect of the invention, the ultrasonic imaging apparatus in the eighth aspect is further provided with an input unit for setting in the tissue part image acquired in advance a marker area which is an area for detecting the kinetic information.

According to this ninth aspect of the invention, an area convenient for detecting kinetic information is selected in the tissue part image.

In the ultrasonic imaging apparatus according to a tenth aspect of the invention, in the ninth aspect the kinetic information is extent of shift information on the shift of the marker area from the point of time of the setting in the B mode image.

According to this tenth aspect of the invention, any shift of the marker area is compensated for.

In the ultrasonic imaging apparatus according to an eleventh aspect of the invention, in the ninth aspect the extent of shift information is information on the extent of parallel shift of the image of the marker area in the B mode image.

According to this eleventh aspect of the invention, the extent of shift information can be found in a simple way.

In the ultrasonic imaging apparatus according to a twelfth aspect of the invention, in any of the ninth through eleventh aspects the kinetic information detecting unit figures out the kinetic information on the basis of the shape of the tissue part image present in the marker area.

According to this twelfth aspect of the invention, the shift of a characteristically shaped tissue part is figured out.

In the ultrasonic imaging apparatus according to a thirteenth aspect of the invention, in the twelfth aspect the kinetic information detecting unit figures out the position of the shape in the B mode image by correlation calculation.

In the ultrasonic imaging apparatus according to a fourteenth aspect of the invention, in any of the ninth through eleventh aspects the kinetic information detecting unit figures out the kinetic information on the basis of the luminance of the tissue part image present in the marker area.

According to this fourteenth aspect of the invention, the shift of the tissue part characterized by luminance is figured out.

In the ultrasonic imaging apparatus according to a fifteenth aspect of the invention, in any of the eleventh through fourteenth aspects the motion compensating unit performs positional compensation on the image of the image information to cancel the extent of shift of the extent of shift information.

According to this fifteenth aspect of the invention, the marker area of image information is made motionless.

In the ultrasonic imaging apparatus according to a sixteenth aspect of the invention, in any of the tenth through fourteenth aspects the motion compensating unit, when displaying the image information on a display unit, performs positional compensation to cancel the extent of shift of the extent of shift information in the displayed position of the display.

According to this sixteenth aspect of the invention, displaying of image information is freed from positional deviations.

According to the invention, image information can be made free from positional deviations by forming a B mode image by using one of a plurality of reflected ultrasonic pulse trains and accurately accomplishing detection of kinetic information on the tissue part using this B mode image and compensation for movements of image information using this kinetic information, and accordingly a synthesized image formed by synthesizing the image information can be made free from blurring and satisfactory in quality.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the overall configuration of an ultrasonic imaging apparatus.

FIG. 2 is a diagram illustrating transmission and reception in the contrast harmonic B mode.

FIG. 3 is a block diagram showing the image processing unit and the image display control unit.

FIG. 4 is a diagram that illustrates operations of the kinetic information detecting means (part 1).

FIG. 5 is a diagram that illustrates operations of the kinetic information detecting means (part 2).

FIGS. 6(A), 6(B), and 6(C) are diagrams that illustrate operations of the synthesized image forming means.

FIG. 7 is a flow chart of the operations of the control unit in the mode for implementation.

FIG. 8 is a diagram that illustrates operations of the motion compensating means in the mode for implementation.

FIG. 9 is a diagram that schematically shows a synthesized image having gone through motion compensation in the mode for implementation.

### DETAILED DESCRIPTION OF THE INVENTION

Best modes for implementing the invention to realize an ultrasonic imaging apparatus will be described below with reference to the accompanying drawings. Incidentally, this is nothing to limit the invention.

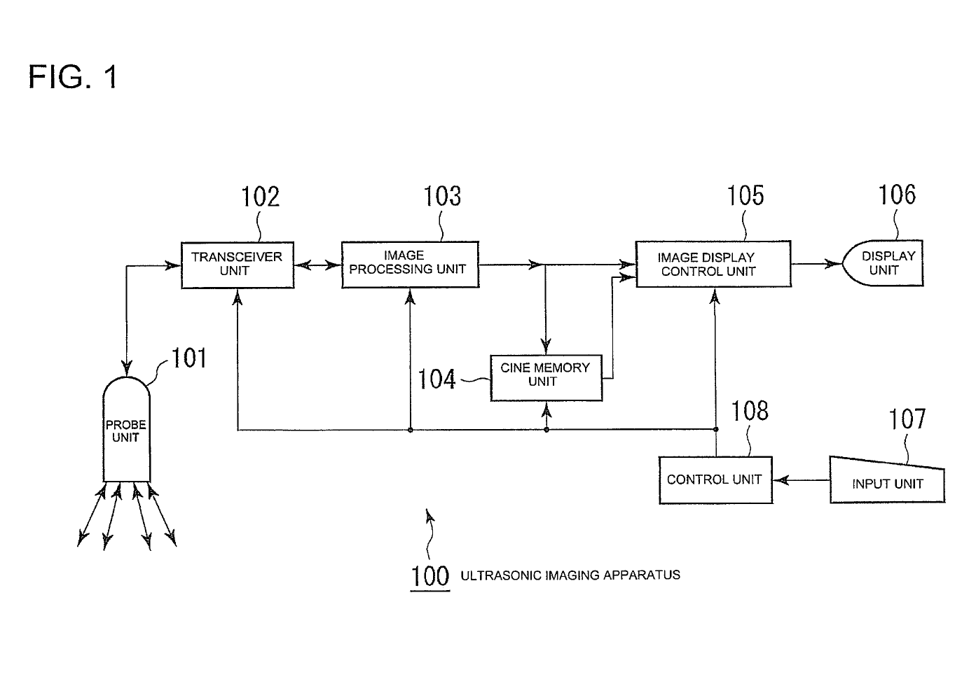

First, the overall configuration of an ultrasonic imaging apparatus 100 in this mode for implementation will be described. FIG. 1 is a block diagram showing the overall configuration of the ultrasonic imaging apparatus 100 in this mode for implementation. The ultrasonic imaging apparatus 100 includes a probe unit 101, a transceiver unit 102, an image processing unit 103, a cine memory unit 104, an image display control unit 105, a display unit 106, an input unit 107 and a control unit 108.

The probe unit 101, which is a part for transmitting and receiving ultrasonic waves, irradiates a subject with ultrasonic pulses and receives reflected ultrasonic pulse trains, which are reflected from time to time from within the subject and constitute a time series. Incidentally, the reflected ultrasonic pulse trains are converted into luminance signals and outputted to the display unit 106 to be described afterwards. The probe unit 101 also is a part for performing electron scanning while successively switching over the irradiating direction of the ultrasonic waves. The probe unit 101 includes an analog multiplexer that selects a probe array, in which piezoelectric elements are arrayed, and the piezoelectric elements, and performs electron scanning.

The transceiver unit 102, connected to the probe unit 101 by a coaxial cable, has pulsers for generating high voltage electric signals for driving the piezoelectric elements of the probe unit 101 and amplifiers for performing the initial stage of amplification of the received reflected ultrasonic pulse trains. The transceiver unit 102 has a plurality of pulsers and an equal number of amplifiers, which are driven substantially simultaneously to perform electron focusing.

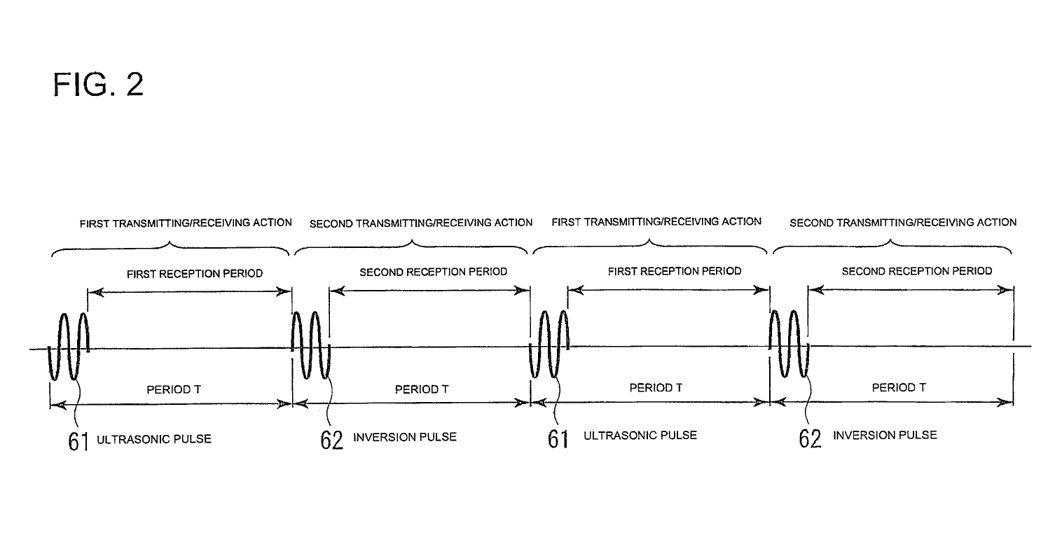

FIG. 2 is a diagram schematically illustrating ultrasonic pulses transmitted from the transceiver unit 102 to the subject when the contrast harmonic B mode is selected by the input unit 107 as an example of contrast imaging mode. In the contrast harmonic B mode, first and second transmitting/receiving actions in which ultrasonic pulses 61 and inversion pulses 62 similar in shape but differing in phase by 180 degrees are transmitted are performed repeatedly, wherein each of the first and second transmitting/receiving actions is completed substantially within a time period T. In the first transmitting/receiving action, after an ultrasonic pulse 61 is transmitted, a reflected ultrasonic pulse trains reflected from the subject is received for a first reception period, and in the second transmitting/receiving action, after an inversion pulse 62 is transmitted, an inversion pulse train reflected from the subject is received for a second reception period. Incidentally, the timings of the ultrasonic pulses 61 and the inversion pulses 62 applied to the piezoelectric elements more or less differ in time from one pulser to another to achieve an electron-focused state in which the phases ultrasonic pulses superpose one another in a prescribed depthwise position within the subject.

The image processing unit 103, including an arithmetic processing unit, a memory and so forth, performs processing to generate a contrast harmonic B mode image and a B mode image on a real time basis from the reflected ultrasonic pulse trains and inversion pulse trains amplified by the transceiver unit 102. Specific contents of this processing include one line of sound ray information by performing A/D (analog/digital) conversion processing, delay and add processing and so forth of the reflected ultrasonic pulse trains and inversion pulse trains that have been received, and processing to write this sound ray information into the image display control unit 105 or the cine memory unit 104 to be described afterwards.

The cine memory unit 104 is an image memory which stores contrast harmonic B mode images and B mode images together with time information, and stores image information formed by the image processing unit 103.

The image display control unit 105 performs display frame rate conversion and control of the shape, position and other factors of image displaying on image information generated by the image processing unit 103 or image information stored in the cine memory unit 104, and displays the image on the display unit 106.

The display unit 106, made up of a CRT (Cathode Ray Tube) or an LCD (Liquid Crystal Display), displays contrast harmonic B mode images, B mode images and so forth.

The input unit 107, made up of a keyboard, a track ball or the like, enables the operator to input control information and so forth. For instance, the input unit 107 is used for inputting control information for performing selection of the contrast harmonic B mode or the B mode, or is used for inputting setting information for setting an ROI (Region Of Interest) on the displayed image displayed on the display unit 106.

The control unit 108 controls the actions of the above-described units of the ultrasonic imaging apparatus in accordance with the control information inputted from the input unit 107 and programs and data stored in advance.

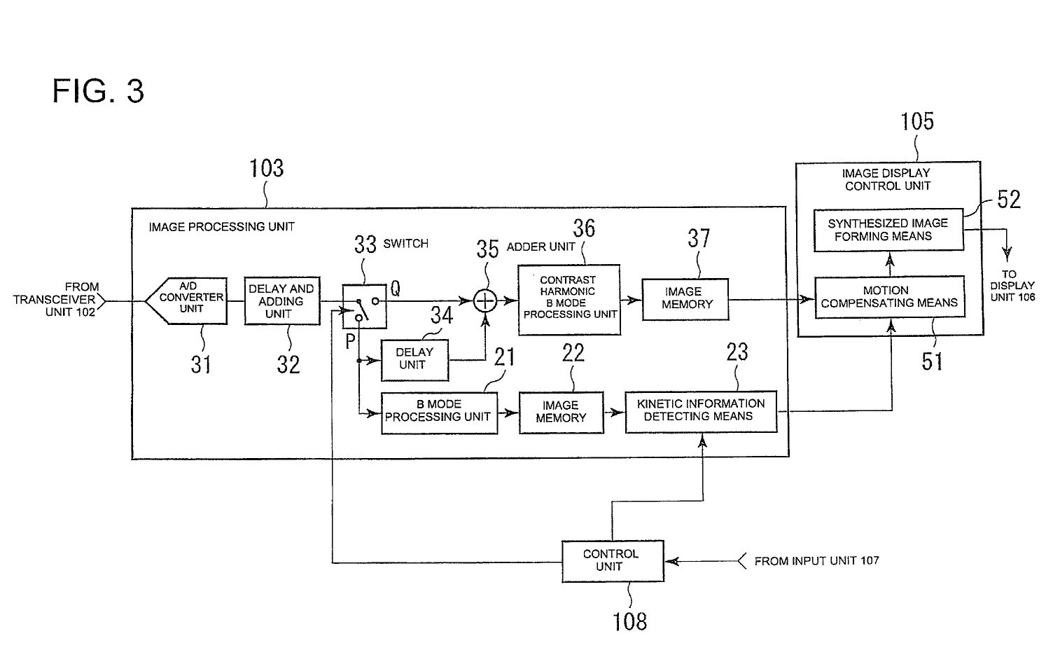

FIG. 3 is a functional block diagram showing the functional configurations of the image processing unit 103 and the image display control unit 105. The image processing unit 103 includes an A/D converter unit 31, a delay and adding unit 32, a switch 33, a delay unit 34, an adder unit 35, a contrast harmonic B mode processing unit 36, an image memory 37, a B mode processing unit 21, an image memory 22 and kinetic information detecting means 23, wherein the image display control unit 105 includes motion compensating means 51 and synthesized image forming means 52.

The A/D converter unit 31 converts the reflected ultrasonic pulse trains and inversion pulse trains, which are analog signals transmitted by the transceiver unit 102, into digital signals during the first and second reception periods shown in FIG. 2. The A/D converter unit 31 has as many A/D converters as transceivers present in the transceiver unit 102, and this number constitutes the largest number of transmitting/receiving actions that can be accomplished by the probe unit 101 at a time.

The delay and adding unit 32 delays and adds reflected ultrasonic pulse trains and inversion pulse trains from the subject, received by a plurality of piezoelectric elements, and electron-focuses them on the prescribed depthwise position within the subject. And the delay and adding unit 32 generates reflected ultrasonic pulse trains and inversion pulse trains arrayed on the time axis corresponding to one line of sound ray.

The switch 33, when performing the first transmitting/receiving action in accordance with a control signal from the control unit 108, connects the output of the delay and adding unit 32 to a P terminal which connects to the delay unit 34 and the B mode processing unit 21 or, when performing the second transmitting/receiving action, connects the output of the delay and adding unit 32 to a Q terminal which connects to the adder unit 35.

The delay unit 34 and the adder unit 35 delay the reflected ultrasonic pulse train received in the midst of the first reception period by the periods T of the first and second transmission/reception and add them to the inversion pulse train received in the second reception period. The ultrasonic pulse 61 and the inversion pulse 62 here differ in phase by 180 degrees, and the linear response part of the added ultrasonic pulse train is deleted, leaving only the nonlinear response part. Incidentally, a contrast medium contains much of high order harmonic components that are nonlinearly responsive to reflected ultrasonic waves. Therefore, from a contrast harmonic B mode image picked up from a subject into that a contrast medium is injected, the contrast medium can be satisfactorily extracted. On the other hand, the tissue part from reflected ultrasonic waves is deleted by adding, and depicted as an unclear image.

The contrast harmonic B mode processing unit 36 and the B mode processing unit 21 applies to the reflected ultrasonic pulse trains and the inversion pulse trains logarithmic conversion and the like to compensate for ultrasonic attenuation according to the reflection depth position within the subject, and configures in the image memory 37 and the image memory 22 a frame which is a sheet of image information on which a plurality of lines of sound line information are arrayed. To add, the contrast harmonic B mode processing unit 36 and the B mode processing unit 21 also performs contrast adjustment and the like. Further, memories of the cine memory unit 104 can as well be used as the image memory 37 and the image memory 22.

The kinetic information detecting means 23 acquires, on the basis of B mode image information stored in the image memory 22, kinetic information on the tissue part that the subject has. As a B mode image depicts the tissue part of the subject whose acoustic impedance is uneven all over as a clear image, it is suitable for detecting the kinetic state of the tissue part.

The kinetic information detecting means 23, for instance, designates from the input unit 107 a marker area on a B mode image displayed on the display unit 106, and detects the motions of the tissue part in this marker area.

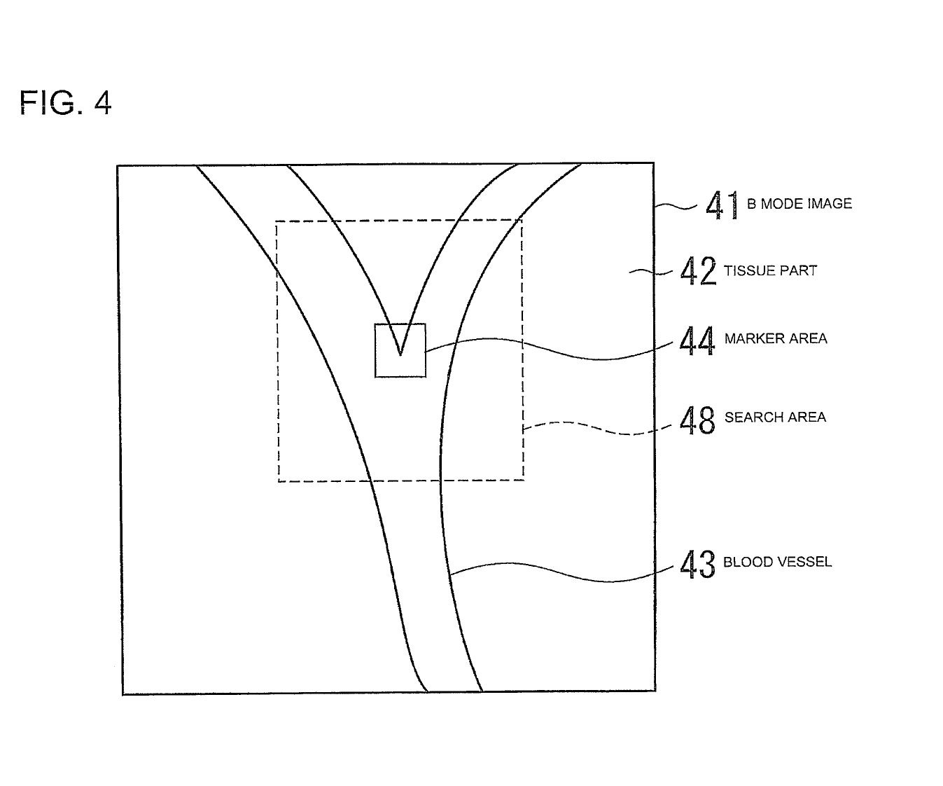

The operator draws a B mode image of the region to be imaged, and sets a marker area in a characteristically shaped part of this frozen B mode image. FIG. 4 shows an example of a marker area 44 in a B mode image 41. This B mode image 41 is an image in which a blood vessel 43 branches and runs in a tissue part 42. Here, for instance, a protruding portion of the tissue part 42 present in the branching point of the blood vessel 43 is selected as an area having a characteristic shape effective for detecting the kinetic state of the tissue part 42. In this selection, the protruding portion is surrounded by the marker area 44, and set and registered as a reference image by key inputting from the input unit 107 or otherwise.

The kinetic information detecting means 23, using the central position of this marker area 44 as the reference position and the shape of the tissue part surrounded by the marker area 44 as the reference image, seeks for kinetic information. As the motion the kinetic information detecting means 23 intends to detect here is a small bodily motion attributable to the pulsation of the blood vessel or the like, the distortion, rotation and other features of the shape of the tissue part 42 designated as the first approximation in the marker area 44 are ignored as being trivial, and only the magnitude of the parallel shift is figured out.

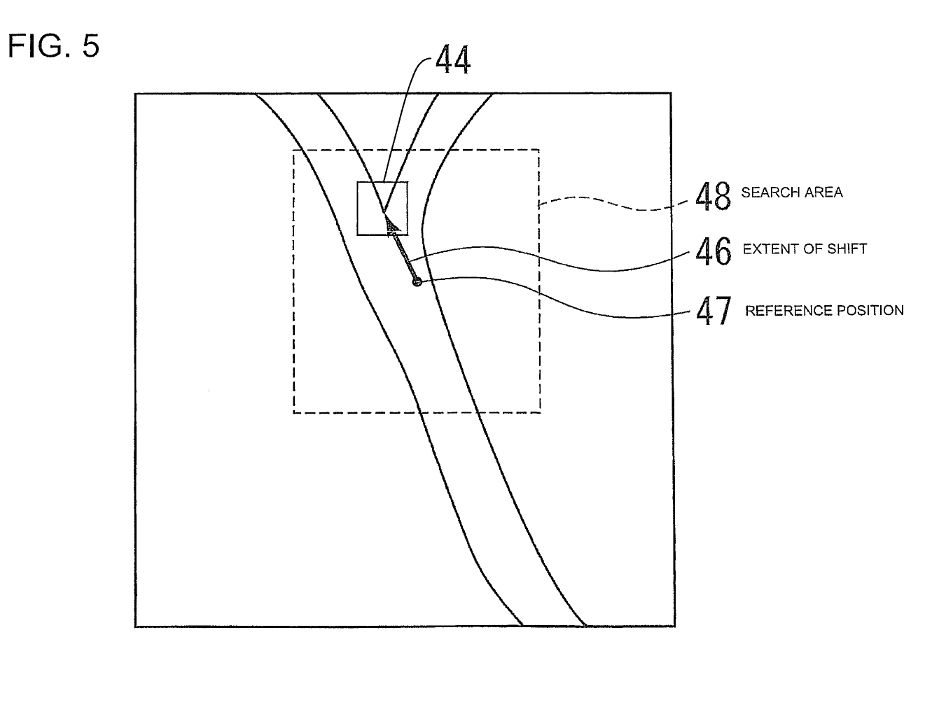

Also, the kinetic information detecting means 23 either manually or automatically sets a search area 48 to search for the shape of the tissue part 42 designated by the marker area 44. The search area 48 represented by broken lines in FIG. 4 is supposed to be large enough to contain the shifting range of the marker area 44 in which the setting has been done. And the kinetic information detecting means 23 detects the position of the reference image which is designated by the marker area 44 and is present in this search area 48, and selects as the kinetic information the parallel shifting distances in the horizontal direction and the vertical direction from the initially set reference position. FIG. 5 shows a case in which the protruding portion of the reference image has moved obliquely upward. The marker area 44 shifts along with the shifting of the reference image, and the extent of its shift 46 from the reference position 47 is represented by an arrow.

Incidentally, as by one of the methods of detecting the position of the reference image in the search area 48, for instance, scanning is performed within the search area 48 while keeping track of correlation in the reference image, and the position of the highest correlation is chosen as the position of the reference image. And the position of the parallel shift is figured out from the difference between this position and the reference position 47, which is the initial position of the reference image.

Referring back to FIG. 3, the synthesized image forming means 52 of the image display control unit 105 compares a plurality of frames of image information, finds the highest of the pixel values in the same pixel position, and forms synthesized image information consisting of a single frame having this highest value as the pixel value in this pixel position. Incidentally, when the plurality of frames of image information to be compared include tomographic image information in the same imaging position varying over time, the synthesized image information integrally represents the variations of the tomographic image information over time (also called cine capture accumulation processing).

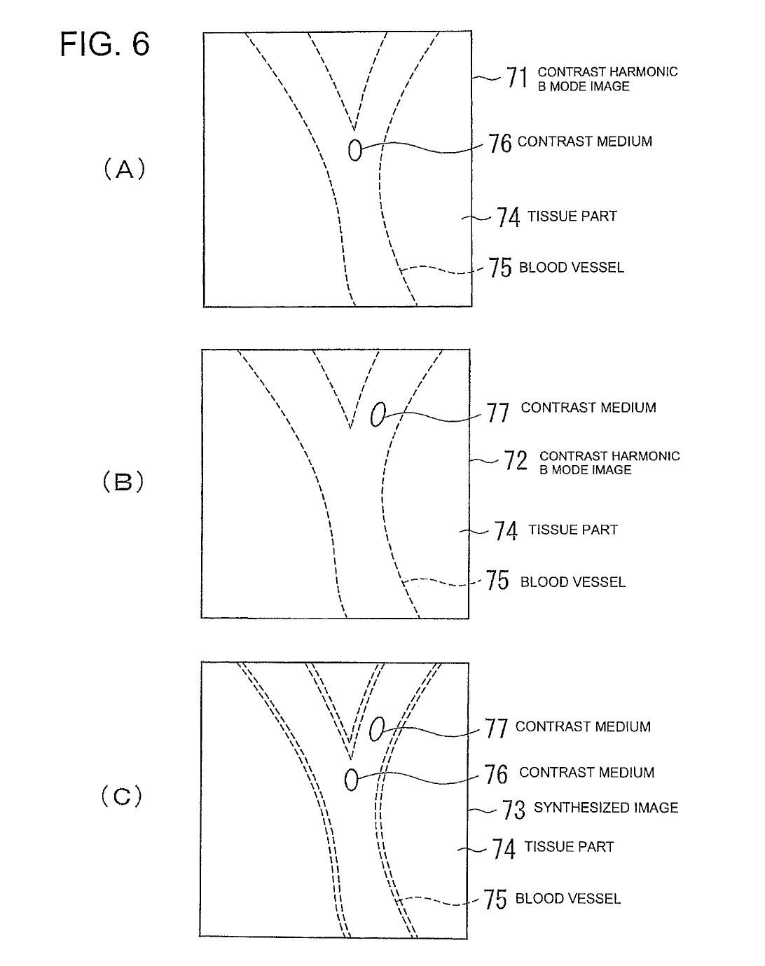

FIGS. 6(A), 6(B), and 6(C) show an example of synthesized image information acquired by using the contrast harmonic B mode when a contrast medium is administered to the subject. Incidentally in the contrast harmonic B mode, the contrast medium in the blood is depicted as a granular high luminance area. FIGS. 6(A) and 6(B) show examples of contrast harmonic B mode images in which the contrast medium in blood flowing in a blood vessel is picked up with a time difference.

FIG. 6(A) schematically illustrate a contrast harmonic B mode image 71 of a blood vessel 75 which is in a tissue part 74 of the subject and branches into two. In the contrast harmonic B mode image 71, as the tissue part 74 is depicted unclearly, the blood vessel 75 which represents the boundary between the tissue part 74 and the blood stream is indicated by broken lines. A contrast medium 76 that flows within the blood is clearly depicted as a granular high luminance area.

FIG. 6(B) shows a contrast harmonic B mode image 72 in which the tissue part 74 and the blood vessel 75 similar to those in FIG. 6(A) are picked up with a time difference. The contrast medium 76 imaged as shown in FIG. 6(A) shifts within the blood vessel 75 along with the blood stream and depicted as a contrast medium 77.

FIG. 6(C) illustrates a synthesized image 73 of the contrast harmonic B mode images 71 and 72. The tissue part 74 and the blood vessel 75 are depicted in the same way as in the contrast harmonic B mode images 71 and 72. The high luminance contrast media 76 and 77 whose positions vary with the flow of blood are both depicted. The images of the blood vessel 75 here are not overlapped with each other by the positional deviations of the contrast harmonic B mode images 71 and 72 acquired with a time difference due to pulsation and other causes, and become blurred. And the synthesized image 73 becomes an image in which the granular contrast medium is shifted from one spot to next by synthesizing a plurality of contrast harmonic B mode images for an extended period of time, namely what traces the shifting of the contrast medium.

The motion compensating means 51 is intended to accurately correct positional deviations of the acquired contrast harmonic B mode image and thereby to prevent the blurring of the synthesized image noted above. Incidentally, details of the motion compensating means 51 will be described below in connection with the operations of the control unit 108.

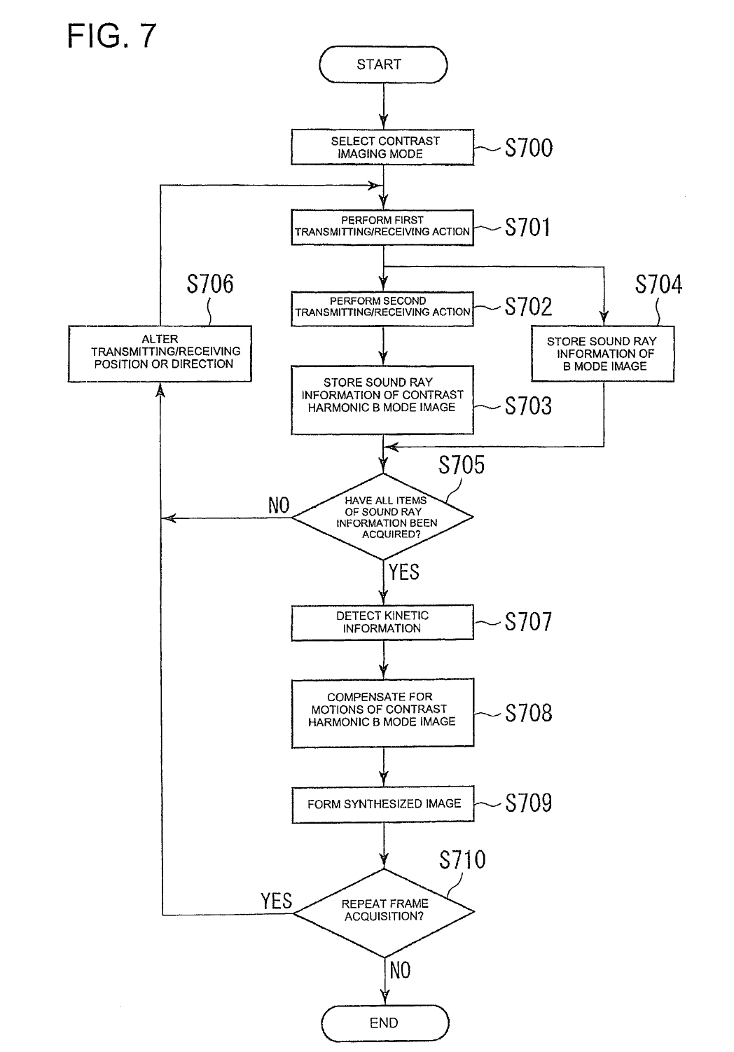

FIG. 7 is a flow chart of the operations of the ultrasonic imaging apparatus 100 in this mode for implementation. First, the operator selects the contrast imaging mode (step S700), and sets into the control unit 108 transmission/reception of the contrast harmonic B mode of the pulse inversion system.

After that, the control unit 108 connects the switch 33 of the image processing unit 103 to the P terminal, and performs the first transmitting/receiving action illustrated in FIG. 2 (step S701). In this procedure, the reflected ultrasonic pulse trains received in the first reception period are inputted to the delay unit 34, and delayed by the period T of the ultrasonic pulse. At the same time, these reflected ultrasonic pulse trains are inputted to the B mode processing unit 21 to cause sound ray information of the B mode image to be produced and stored into the image memory 22 (step S704).

After that, the control unit 108 connects the switch 33 of the image processing unit 103 to the Q terminal, and performs the second transmitting/receiving action illustrated in FIG. 2 (step S702). In this procedure, the inversion pulse trains received in the second reception period are added, by the adder unit 35, to the reflected ultrasonic pulse trains received in the first reception period and outputted from the delay unit 34, and inputted to the contrast harmonic B mode processing unit 36. In the contrast harmonic B mode processing unit 36, sound ray information of the contrast harmonic B mode image is produced and stored into the image memory 37 (step S703).

After that, the control unit 108 determines whether or not all the items of sound ray information constituting one frame have been acquired (step S705) and, if all the items of sound ray information have not been acquired (NO at step S705), the transmitting/receiving position or the transmitting/receiving direction of the piezoelectric elements arrayed in the probe unit 101 is altered (step S706), followed by a shift to step S701 to repeat the first transmitting/receiving action and the second transmitting/receiving action.

The control unit 108, if all the items of sound ray information constituting one frame have been acquired (YES at step S705), detects kinetic information with the kinetic information detecting means 23 by using B mode image information stored in the image memory 22 (step S707). This kinetic information includes information on the extent of shift 46 of the marker area 44 set in advance in the B mode image.

After that, the motion compensating means 51 compensates for the motions of the contrast harmonic B mode image on the basis of the contrast harmonic B mode image information inputted from the image memory 37 and the information on the extent of shift 46 inputted from the kinetic information detecting means 23 (step S708).

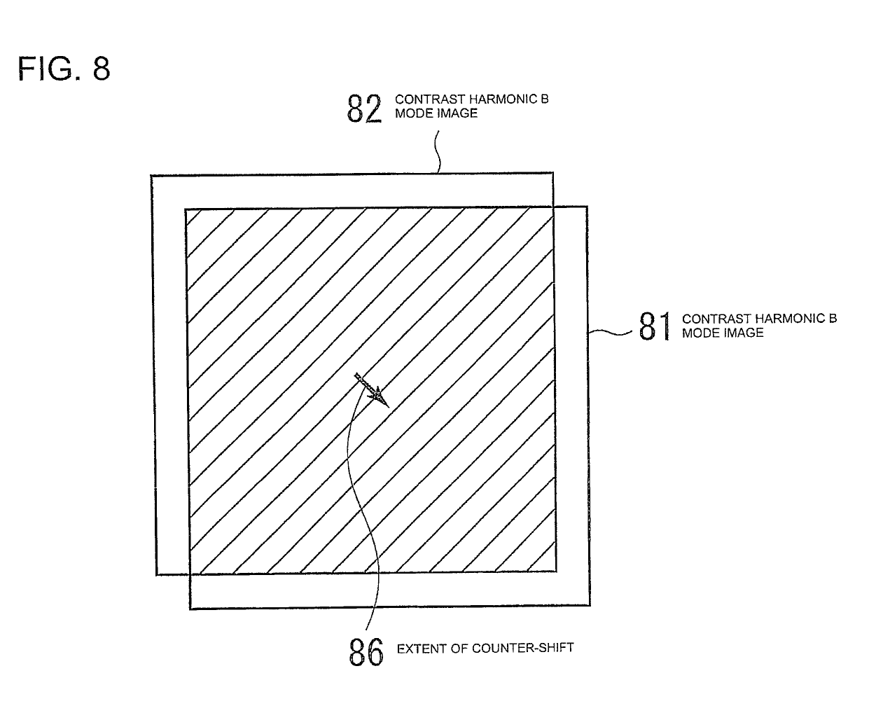

FIG. 8 schematically illustrates the compensation for motions performed by the motion compensating means 51. The motion compensating means 51 forms a new contrast harmonic B mode image 82 resulting from shifting of a contrast harmonic B mode image 81 acquired from the image memory 37 by an extent of counter-shift 86 which differs in shifting direction by 180 degrees from the extent of shift 46 detected by the kinetic information detecting means 23. This causes the shifting of the image parts designated by the marker area 44 to be substantially cancelled and these parts become still. Incidentally, in forming the new contrast harmonic B mode image 82, as a part deficient in sound ray information arises according to the magnitude of the extent of counter-shift 86, for instance zero-value data are read into this part or, if discrepancy in sound ray position occurs, data are interpolated or some other measure is taken.

After that, the control unit 108 forms a synthesized image on the basis of the contrast harmonic B mode image 82 whose motions have been compensated (step S709). In this synthesized image formation, as the reference image in the area designated by the marker area 44 is placed in a substantially still state, the synthesized image for depicting contrast media as shown in FIG. 6 is free from blurring.

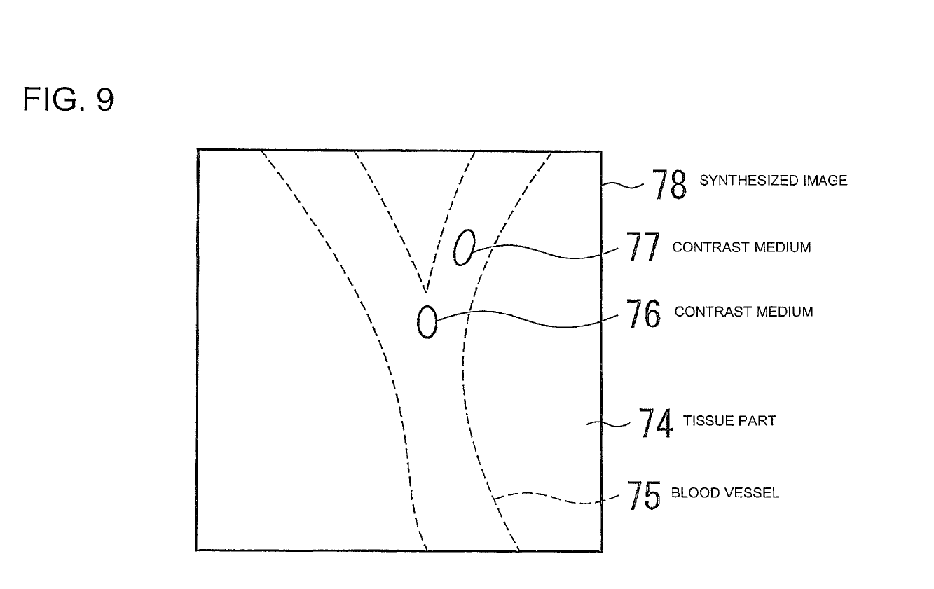

FIG. 9 schematically shows a synthesized image 78 synthesized after the motions of the contrast harmonic B mode images 71 and 72 shown in FIGS. 6(A) and 6(B) have been compensated for. The blood vessel 75 in the synthesized image 78 becomes clear, particularly free from blurring due to positional deviations between the contrast harmonic B mode images 71 and 72. Further, the synthesized image 78 is made accurate, cleared of positional deviations between the contrast media 76 and 77 in the blood vessel 75.

After that, the control unit 108 determines whether or not to repeat frame acquisition (step S710) and, if frame acquisition is to be repeated (YES at step S710), the processing shifts to step S701 to repeat transmission/reception of the drive pulse. If frame acquisition is not to be repeated (NO at step S710), the control unit 108 ends this processing.

As hitherto described, in this mode for implementation, when the contrast harmonic B mode is selected and transmission/reception is to be performed by the pulse inversion system, the reflected ultrasonic pulse trains received by the first transmitting/receiving action are inputted not only to the delay unit 34 but also to the B mode processing unit 21 to generate the B mode image at the same time as the contrast harmonic B mode image, detects motion information on the tissue part of the subject on the basis of this B mode image, and corrects the position of the image by using this motion information when forming a synthesized image of the contrast harmonic B mode image; therefore, it is possible to securely compensate the motions of the synthesized image using the contrast harmonic B mode image while maintaining the frame rate of the contrast harmonic B mode image and without sacrificing real time availability, enabling a blur-free synthesized image to be formed.

Further in this mode for implementation, when kinetic information on the subject is to be detected by using the kinetic information detecting means 23, the characteristically shaped tissue part 42 is supposed to be designated as the marker area 44, but it is also possible to designate the high luminance area of the tissue part 42 as the marker area 44 and to detect the motion of this high luminance area by luminance peak detection or otherwise.

Further in this mode for implementation, when the first transmitting/receiving action is to be performed, the reflected ultrasonic pulse trains in the first reception period are supposed to be used as sound ray information on the B mode image, but similarly sound ray information on the B mode image can be generated by using the inversion pulse trains received in the second reception period when the second transmitting/receiving action is to be performed.

Further in this mode for implementation, though one line of sound ray information for forming image information is supposed to be generated after going through the first and second reception periods, one line of sound ray information may as well be generated after a plurality each of first and second reception periods. In this case, too, sound ray information of the B mode image can be similarly generated by using the reflected ultrasonic pulse trains or the inversion pulse trains acquired by any transmitting/receiving action.

Further in this mode for implementation, though it is supposed to correct the position of contrast harmonic B mode image to form the synthesized image 78, positional correction can as well be performed by using the detected extent of shift 46 or the like to obtain an image free from positional deviations when displaying the contrast harmonic B mode images 71, 72, 81 and so forth on the display unit 106.

Further in this mode for implementation, though it is supposed to compensate the motions of the contrast harmonic B mode image with the motion compensating means 51 of the image display control unit 105, the motion compensating means 51 can as well be shifted to the image processing unit 103 to compensate the motions there.

Further regarding this mode for implementation, an example of contrast harmonic B mode of the pulse inversion system used when in the contrast imaging mode was cited, similar motion compensation can also be achieved in another mode in which a plurality of driving pulses are used to acquire one line of sound ray information. For instance, similar motion compensation can be achieved also in the case of using a system of coded excitation in which the driving pulses to be applied to the piezoelectric elements is as the driving pluses coded and having time differences when transmitting the ultrasonic pulse.

Many widely different embodiments of the invention may be configured without departing from the spirit and the scope of the present invention. It should be understood that the present invention is not limited to the specific embodiments described in the specification, except as defined in the appended claims.

1. An ultrasonic imaging apparatus configured to transmit a plurality of ultrasonic pulses to a subject, receive a plurality of reflected ultrasonic pulse trains reflected from the subject, generate a line of sound ray information by using the plurality of reflected ultrasonic pulse trains, and to form image information in which items of the line of sound ray information differ according to one of a position and a direction of the transmission of the plurality of ultrasonic pulses and the reception of the plurality of reflected ultrasonic pulse trains are arrayed, said ultrasonic imaging apparatus comprising:
a B mode processing unit configured to form B mode image information by using any of the plurality of reflected ultrasonic pulse trains;
a kinetic information detecting unit configured to detect kinetic information of the subject based on the B mode image information; and
a motion compensating unit configured to compensate image information for motions based on the kinetic information.

2. An ultrasonic imaging apparatus configured to:
perform a first transmitting/receiving action in which a first set ultrasonic pulses are transmitted to a subject and a first reflected ultrasonic pulse train associated with the first set of ultrasonic pulses is received;
perform a second transmitting/receiving action in which a second set of ultrasonic pulses similar in shape but differing in phase by 180 degrees from the first set of ultrasonic pulses are transmitted to the subject and a second reflected ultrasonic pulse train associated with the second set of ultrasonic pulses is received;
generate a line of sound ray information based on the first reflected ultrasonic pulse train and the second reflected ultrasonic pulse train; and
form image information in which items of the line of sound ray information differ according to one of a position and a direction of the first transmitting/receiving action and the second transmitting/receiving action are arrayed, wherein said ultrasonic imaging apparatus comprises:
a B mode processing unit configured to form B mode image information based on one of the first reflected ultrasonic pulse train and the second reflected ultrasonic pulse train;
a kinetic information detecting unit configured to detect kinetic information of the subject based on the B mode image information; and
a motion compensating unit configured to compensate image information for motions based on the kinetic information.

3. The ultrasonic imaging apparatus according to claim 2, wherein the first transmitting/receiving action and the second transmitting/receiving action are performed a plurality of times each to generate the line of sound ray information.

4. The ultrasonic imaging apparatus according to claim 2, wherein the line of sound ray information is generated by adding the first reflected ultrasonic pulse train and the second reflected ultrasonic pulse train.

5. The ultrasonic imaging apparatus according to claim 1, further comprising a synthesized image forming unit configured to compare pixel values in a same pixel position in a plurality of sets of image information formed in a time sequence, and to form synthesized image information based on a highest pixel value.

6. The ultrasonic imaging apparatus according to claim 5, wherein said synthesized image forming unit is configured to form the synthesized image information by using image information having undergone motion compensation.

7. The ultrasonic imaging apparatus according to claim 1, further comprising a memory unit configured to store, in a time series, the image information together with time information that has been formed.

8. The ultrasonic imaging apparatus according to claim 1, wherein said kinetic information detecting unit is configured to detect the kinetic information on the tissue part image of the subject contained in the B mode image of the B mode image information.

9. The ultrasonic imaging apparatus according to claim 8, further comprising an input unit configured to set in the tissue part image acquired in advance a marker area which is an area for detecting the kinetic information.

10. The ultrasonic imaging apparatus according to claim 9, wherein the kinetic information is extent of shift information based on a shift of the tissue part image within the marker area from the point of time of setting the marker area in the B mode image.

11. The ultrasonic imaging apparatus according to claim 10, wherein the extent of shift information is based on an extent of parallel shift of the tissue mart image within the marker area in the B mode image.

12. The ultrasonic imaging apparatus according to any of claim 9, wherein said kinetic information detecting unit is configured to determine the kinetic information based on a shape of the tissue part image present in the marker area.

13. The ultrasonic imaging apparatus according to claim 12, wherein said kinetic information detecting unit is configured to determine a position of the shape in the B mode image using a correlation calculation.

14. The ultrasonic imaging apparatus according to claim 9, wherein said kinetic information detecting unit is configured to determine the kinetic information based on a luminance of the tissue part image present in the marker area.

15. The ultrasonic imaging apparatus according to claim 10, wherein said motion compensating unit is configured to perform a positional compensation on the image of the image information to cancel the extent of shift of the extent of shift information.

16. The ultrasonic imaging apparatus according to claim 10, wherein said motion compensating unit, when displaying the image information on a display unit, is configured to perform a positional compensation to cancel the extent of shift of the extent of shift information in a displayed position of said display.

17. An ultrasonic imaging method comprising:
transmitting a plurality of ultrasonic pulses to a subject;
receiving a plurality of reflected ultrasonic pulse trains reflected by the subject;
generating a line of sound ray information based on the plurality of reflected ultrasonic pulse trains;
forming B mode image information using based on at least one of the plurality of reflected ultrasonic pulse trains;
detecting kinetic information of the subject based on the B mode image information;
forming image information in which items of the line of sound ray information differ according to one of a position and a direction of the transmitted plurality of ultrasonic pulses and the received plurality of reflected ultrasonic pulse trains; and
compensating the image information for motion based on the kinetic information.

18. An ultrasonic imaging method in accordance with claim 17, further comprising:
comparing pixel values in a same pixel position in a plurality of sets of image information formed in a time sequence; and
forming synthesized image information including a highest pixel value.

19. An ultrasonic imaging method in accordance with claim 18, wherein forming synthesized image information comprises forming synthesized image information using image information having undergone motion compensation.

20. An ultrasonic imaging method in accordance with claim 17, further comprising storing, in a time series, the image information and time information that has been formed.
